(19) United States
(12) Patent Application Publication
(10) Pub. No.: US 2009/0120834 A1
(43) Pub. Date: May 14, 2009

(54) Sustainability in personal care product retailing

(75) Inventors: Marcille Faye Ruman, Oshkosh, WI (US); Heidi Ellen Vanden Boom, Appleton, WI (US); Paula Sosalla, Appleton, WI (US); Heidi Bauerlein Hopkins, Neenah, WI (US); Karissa Brummond, Mayville, WI (US); Abayomi Michael Shonoiki, Combined Locks, WI (US); Margaret Mary Posorske, Oshkosh, WI (US)

Correspondence Address: KIMBERLY-CLARK WORLDWIDE, INC.;Catherine E. Wolf, 401 NORTH LAKE STREET, NEENAH, WI, 54956, US

(73) Assignee: Kimberly-Clark Worldwide, Inc.

(21) Appl. No.: 11/983,840

(22) Filed: Nov. 13, 2007

(51) Int. Cl. B65D 79/00; B65D 5/52; A61B 19/02; B65D 73/00

(52) U.S. Cl. 206/745; 206/736; 206/438; 206/494

(57) ABSTRACT

An array of packages adapted for transporting and selling personal care products is provided. The array includes a first package including first removable product-identifying graphics and a plurality of first personal care products disposed within a first storage bin, the first storage bin including a first design and being substantially free of product-identifying graphics. The array also includes a second package including second removable product-identifying graphics and a plurality of second personal care products disposed within a second storage bin, the second storage bin including a second design and being substantially free of product-identifying graphics, wherein the second design is different from and complementary to the first design. The first and second personal care products can be different sizes of the same product, different products, the same product but adapted to be sold at differently-branded retailers, or different combinations of personal care products.

### BACKGROUND

Personal care products such as toiletries, moist and dry wipes, diapers, training pants, incontinence products, feminine hygiene products, swim undergarments, and the like, are conventionally packaged in discardable packaging, including significant portions that are not or cannot be recycled.

Manufacturers, retailers, and consumers of personal care products recognize a need to address environmental concerns. One slogan used to promote environmental awareness and sustainability is reduce, reuse, and recycle. Current personal care product packaging schemes, however, cannot meet these goals. An opportunity exists to create extended-use packaging to help meet these goals.

### SUMMARY

The goals of reducing, recycling, and reusing can be accomplished in packaging used to deliver products to a consumer by reducing the amount of packaging for a given product, making the packaging useful and appealing enough to promote their reuse, and by using packaging materials that are recyclable. The present disclosure describes packaging that can accomplish these goals.

The present inventors undertook intensive research and development efforts concerning improving the delivery of personal care products, particularly with respect to providing packaging that can be recycled and/or reused. The packaging not only serves to contain and identify personal care products for shipping and retail purposes, but converts the packaging into another use that is tested to be highly desired by the consumer. The disclosure presented herein demonstrates personal care product packaging in the form of a reusable storage bin. After emptying the personal care product contents, a consumer can use the handy, stackable storage bins, thus extending the packaging usefulness to the consumer.

An array of packages adapted for transporting and selling personal care products is provided. The array includes a first package including first removable product-identifying graphics and a plurality of first personal care products disposed within a first storage bin, the first storage bin including a first design and being substantially free of product-identifying graphics. The array also includes a second package including second removable product-identifying graphics and a plurality of second personal care products disposed within a second storage bin, the second storage bin including a second design and being substantially free of product-identifying graphics, wherein the second design is different from and complementary to the first design. The first and second personal care products can be different sizes of the same product, different products, the same product but adapted to be sold at differently-branded retailers, or different combinations of personal care products.

### BRIEF DESCRIPTION OF THE DRAWINGS

The foregoing and other features and aspects of the present disclosure and the manner of attaining them will become more apparent, and the disclosure itself will be better understood by reference to the following description, appended claims, and accompanying drawing.

FIG. 1 is a partially-cutaway perspective view of one aspect of a storage bin.

Repeat use of reference characters in the present specification and drawing is intended to represent the same or analogous features or elements of the present disclosure.

### DETAILED DESCRIPTION

It is to be understood by one of ordinary skill in the art that the present discussion is a description of exemplary aspects only, and is not intended as limiting the broader aspects of the present disclosure.

The present disclosure is generally directed to personal care products and how they are packaged for transport and retail sale. The personal care product can be, for instance, a moist or dry wipe, a toiletry, or an absorbent article such as a diaper, a training pant, an incontinence product, a feminine hygiene product, a medical garment, a bandage, or the like. Generally, the personal care products are disposable, meaning that they are designed to be discarded after a limited use rather than being laundered or otherwise restored for reuse.

Manufacturers, retailers, and consumers of personal care products recognize a need to address environmental concerns. One slogan used to promote environmental awareness and sustainability is reduce, reuse, and recycle. These goals can be accomplished in packaging used to deliver products to a consumer by reducing the amount of packaging for a given product, making the packaging useful and appealing enough to promote their reuse, and by using packaging materials that are recyclable. The present disclosure describes packaging that can accomplish these goals.

The packaging described herein is a value added and retailer-differentiated solution. The packaging is unique in that it includes attractive graphics and branding to convey that the storage bin is an easy-organizing extra for the customer, shopper, and/or user, and is worth a higher price per personal care product. The storage bin itself carries minimal to no information. Instead, a removable bin enclosure communicates all of the manufacturing, distribution, branding, and labeling requirements. A package violator can communicate product claims with respect to reusable storage, stylish design, and environmental sustainability.

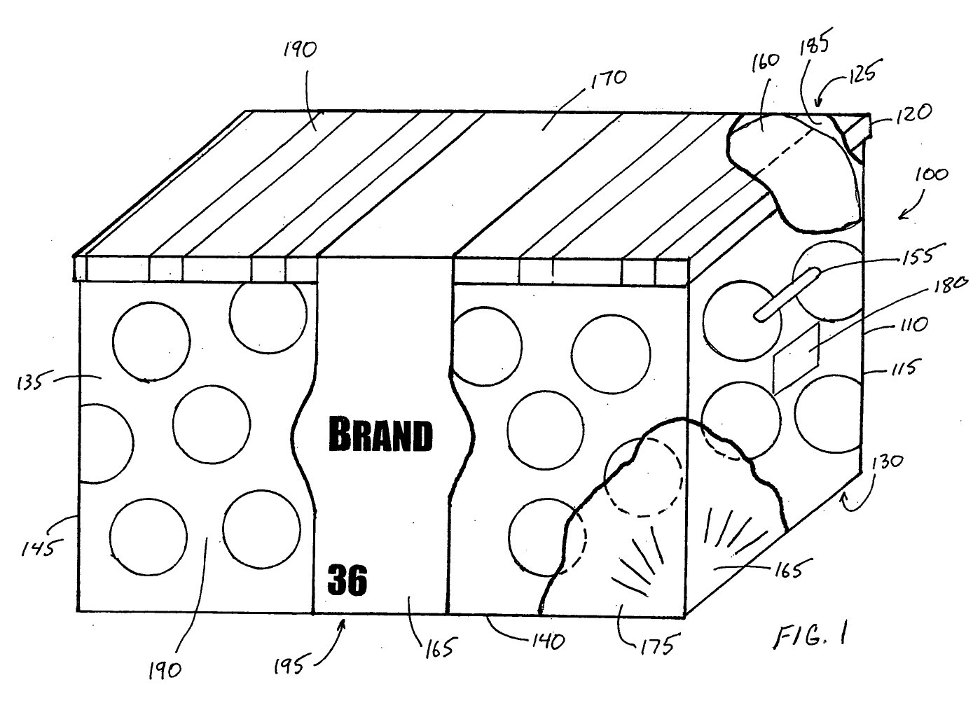

The packaging described herein is particularly useful for transporting and selling disposable personal care products including absorbent articles, wipes, toiletries, and other personal health and hygiene items. Referring to FIG. 1 for exemplary purposes, a packaging system 100 is shown. The packaging system 100 is adapted such that all or most of the packaging system 100 is readily reused and/or recycled. The packaging system 100 described herein increases the likelihood a consumer will select more environmentally sustainable packaging for disposable personal care products, thus eliminating the need for standard packaging.

As illustrated in FIG. 1, the packaging system 100 includes a storage bin 110 as its central component. The storage bin 110 includes a storage bin bottom 115 and a cover 120. The cover 120 is either partially or completely removable from the storage bin bottom 115, and is capable of being reattached to or re-set upon the storage bin bottom 115 to reclose the storage bin 110. In other aspects, the cover 120 can be in the form of a single or multiple flaps. The storage bin bottom 115 and cover 120 together define an interior space 125 of the storage bin 110.

The storage bin 110 as illustrated is generally rectilinear, but can be any suitable shape. The storage bin bottom 115 includes a lower face 130 and, if rectilinear, also includes four sides. If, for example, the storage bin 110 is generally cylindrical, the storage bin bottom 115 will include only one side 135 extending around the perimeter of the lower face 130. The storage bin 110 has a footprint, which is generally the view of the lower face 130 either from below or from the interior space 125. If the storage bin 110 is rectilinear, then the footprint will be rectilinear as well. If the storage bin 110 is cylindrical, then the footprint will be circular. The footprint can be any other suitable shape including an oval, a hemisphere, a triangle, a square, or any other shape including linear and/or arcuate portions.

The storage bin 110 also defines a horizontal perimeter 140 and a vertical perimeter 145. The horizontal perimeter 140 is generally the distance around the storage bin 110 on a horizontal plane, such as the perimeter of the lower face 130. The vertical perimeter 145 is generally the distance around the storage bin 110 on a vertical plane, such as across the cover 120, across the lower face 130, and across two opposed sides 135.

The storage bin 110 can be manufactured from any suitable material including corrugated cardboard, injection-molded or other plastic, wood, woven or nonwoven fabrics, or combinations of these. As a result, the storage bin 110 can be rigid or flexible, or a combination of these. Part or all of the storage bin 110 can be opaque, translucent, or generally transparent, and can be of any suitable color. In one example, the storage bin 110 can be manufactured from corrugate sturdy enough to support reuse of the storage bin 110 by the consumer. In another example, the storage bin 110 can be manufactured from multi-layered, insulated, liquid-resistant fabric such that a consumer can reuse the storage bin 110 as a cooler. In still another example, the storage bin 110 can be manufactured from plastic such that the consumer can reuse the storage bin 110 for semi-permanent storage, as a planter, or for any other typical household use.

Packaging used prior to this invention is optimized to minimize material usage; it is designed to be only durable enough to get products to a consumer. As a result, current packaging is not durable and has limited reuse possibilities. In addition, the graphics present on current packaging are targeted for point of sale and therefore do not blend with home and baby décor. Packaging of the present disclosure is made to be more durable and thus have uses beyond shipping and retailing purposes. In addition, point-of-sale graphics are removable to remove the clash between point-of-sale graphics and home décor. Finally, the storage bin 110 can feature home décor graphics, a label area, a removable cover 120, etc. Consumers want ready-to-use packaging; they do not want to assemble a storage bin 110 or a cover 120, and they want easy access to the packaging and product.

There are many storage bin styles including but not limited to, slotted boxes, telescoping boxes, folders, rigid boxes such as Bliss boxes, self-erecting boxes, corrugated common footprint containers, and interior forms. Interior forms such as liners, tubes, pads, build-ups, dividers, partitions, and other inner packaging pieces can be made in an infinite variety of ways to separate or cushion products, to strengthen the storage bin 110, or to fill voids to prevent products from moving.

In the aspects of the present disclosure in which the storage bin 110 is made from corrugate, a consumer-preferred version of a reusable storage bin 110 is a half-slotted storage bin 110 and a tray as the cover 120. Half-slotted means that the storage bin 110 has no top flaps. Interior forms may be added to the storage bin 110 to improve storage bin durability and/or to increase functionality and reusability of the storage bin 110. A Bliss box, which has no bottom flaps and which is similar to a standard shoe box, is also very appealing to consumers because it eliminates gaps and the potential for the bottom to break open.

Also, in the aspects of the present disclosure in which the storage bin 110 is made from corrugate, corrugated fiberboard, or combined board, has two main components: the linerboard and the medium. Both are made of a special kind of heavy paper called container board. Linerboard is the flat facing or liner that adheres to the medium. The medium is the corrugated or fluted paper glued between the linerboard facings. Linerboard can be single face, single wall, double wall, or triple wall. Flutes come in several common sizes or profiles ranging from A (33 flutes/foot) to F (125 flutes/foot). Smaller flute profiles provide enhanced structural and graphics capability for primary and retail packaging, while larger flute profiles deliver a greater vertical compression strength and cushioning. Different flute profiles can be combined in one piece of combined board. Mixing flute profiles allows designers to manipulate the compression strength and total thickness of the combined board.

For storage bins 110 of the present disclosure, a consumer-preferred version of a reusable storage bin 110 is a single- or double-wall structure depending on the substrates (e.g., materials, weight, flute) used. This is done to minimize the storage bin's vulnerability to crushing, abrasion, and puncturing. Consumers desire high-quality graphics on a strong and durable storage bin 110. Retail diaper and wipes boxes commonly use B or C flutes to balance aesthetic and structural requirements. In double wall containers, varied flute profiles provide advantages over flutes of the same size that are perfectly aligned. Smaller and finer fluting is desired on the outside of the storage bin to maintain high quality graphics.

Corrugating adhesive is normally bonded to the liners with a starch-based adhesive. It is available in several levels of water resistance. A flat piece of corrugated fiberboard that has been cut, slotted, and scored is called a box blank. For some box styles, in order to make a box, the two ends of the box blank must be fastened together with glue, tape, or staples. The place where these two ends meet is known as the manufacturer's joint. For storage bins 110 of the present disclosure, a glue joint on the inside of the storage bin 110 is preferred. This is done for both aesthetics and safety reasons. Also, for the storage bin 110 and cover 120 in general, it is desired to have the entire structure glued versus taped or stapled.

Accurate inside storage bin dimensions must be determined to ensure the proper fit of the products being shipped or stored. In addition, accurate outside storage bin dimensions must be determined to optimize palletizing and distributing the product. For storage bins 110 of the present disclosure, storage bin dimensions are influenced by several factors including: consumer-preferred storage bin dimensions, retailer-desired price point of offering, product quantity, product size and configuration (e.g., arrangement and orientation), pallet footprint and cube utilization, and manufacturing equipment limitations.

In other aspects of the present disclosure, windows or special cut outs can be added to the storage bin 110 to enhance functionality or aesthetic appeal, including to add air vents to the storage bin 110. In still other aspects of the present disclosure, the storage bin 110 can include a second storage bin 110 inside for strength.

The storage bin 110 as it is presented to the consumer includes disposable personal care products within the interior space 125 of the storage bin 110. Such disposable personal care products include, but are not limited to, absorbent articles, dry, moist, or wet wipes, toiletries, and other personal care items. Absorbent articles include diapers, training pants, swim pants, incontinence products, feminine hygiene products, bandages, and the like. The personal care products can be disposed within the interior space 125 in any suitable arrangement.

In another aspect of the present disclosure, the personal care products can be disposed within one or more inner packages 160. Such inner packages 160 can provide compression, containment, environmental protection, and/or organization to the personal care products. In one aspect, the inner package 160 is a flexible consumer package, which refers to non-rigid containers, such as polyethylene bags, that are adapted to contain personal care products and are adapted to be presented to a consumer. In other various aspects, the inner package 160 is manufactured from standard poly film, paper, woven or nonwoven fabric, or from any other suitable material or combination of materials and in any suitable manner. In another aspect promoting reuse of the inner package 160, the inner package 160 includes a reclosing element that allows the inner package to be reclosed after it has been opened. Such a reclosing element can be a zipper-type closure or any other suitable closure element. In another aspect, the reclosing element can make the inner package 160 airtight.

The packaging system 100 can also include a bin enclosure 165 that partially or completely envelops the storage bin 110. The bin enclosure 165 can assist in ensuring the cover 120 stays in place atop the storage bin bottom 115 through manufacturing, shipping, and retail handling. The bin enclosure 165 can also provide space on which to provide product, brand, and other labeling information. The bin enclosure 165 can also provide environmental protection to shield the storage bin 110 from dust, dirt, water, and other hazards that might otherwise adversely affect the appearance, performance, and value of the storage bin 110 and the products within.

In one aspect of the present disclosure, the bin enclosure 165 is a sleeve 170 that surrounds all or part of the vertical perimeter 145 of the storage bin 110. In general, the sleeve 170 encircles the storage bin 110 to provide both a means of keeping the cover 120 in place atop the storage bin bottom 115 and an additional surface on which to provide product, brand, and other labeling information. The sleeve 170 has a bin-facing side and an outward-facing side. The sleeve 170 can be manufactured from paperboard, poly film, woven or nonwoven fabric, or any other suitable material or combination of materials. The sleeve 170 is wrapped around the storage bin 110 and then attached to itself and/or the storage bin 110 using adhesives, tabs, or any other suitable mechanisms. The sleeve 170 can be centered on the storage bin 110 or can be offset toward one side of the storage bin 110.

In another aspect of the present disclosure, the bin enclosure 165 is an overwrap 175 that surrounds all or part of the storage bin 110. In general, the overwrap 175 encloses the storage bin 110 to provide a means of keeping the cover 120 in place atop the storage bin bottom 115; an additional surface on which to provide product, brand, and other labeling information; and environmental protection of storage bin 110 and its contents. The overwrap 175 can be manufactured from poly film, polarizing film, woven or nonwoven fabric, or any other suitable material or combination of materials. The overwrap 175 is wrapped around the storage bin 110 and then attached to itself and/or the storage bin 110 using heat, shrinking, adhesives, tabs, or any other suitable mechanisms.

In general, the bin enclosure 165, whether it is a sleeve 170 or an overwrap 175, can include indicia indicating a secondary use of the bin enclosure 165. In addition, the packaging system 100 can include both a sleeve 170 and an overwrap 175. In this aspect, the sleeve 170 is typically placed directly on the storage bin 110 as a means of keeping the cover 120 in place atop the storage bin bottom 115. The overwrap 175 is then placed to enclose both the storage bin 110 and the sleeve 170 as environmental protection of the storage bin 110 and its contents. In this aspect, either or both of the sleeve 170 and the overwrap 175 can be used as a surface on which to provide product, brand, and other labeling information.

With respect to sustainable practices, reduction is at the head of the Environmental Protection Agency's waste management hierarchy: reduce, reuse, recycle, compost and recover energy. Sustainable practices are being promoted across all segments of the packaged goods supply chain. By implementing sustainable packaging such as reusable storage bins 110, society will generate less waste, use fewer materials, and generate savings in manufacturing, transportation, storage, and disposal/recovery. Specific variables that can be controlled include greenhouse gas emissions emitted during manufacture, the environmental-friendliness of materials, product-to-package ratio, cube utilization, transportation impacts, recycled content, recovery value, renewable energy used, and sustainable innovations.

Diapers and other personal care products are already sold in corrugated boxes that consumers tend to recycle or simply discard, to end up in landfills. Also, consumers buy plastic totes to save and store baby things and other items. Shipping and retailing personal care products in a reusable storage bin 110 accrues a double benefit—recycled or discarded shipping boxes are eliminated, and the need to buy additional storage containers is eliminated as well. Consumer and manufacturer efforts are synchronized in getting personal care products to the consumer in a beautiful and earth-friendly storage bin 110.

Additionally, waste is minimized for the selling unit, and the selling unit can be made from earth-friendly materials, recycled, and/or reused. This holds true for the supplementary packaging used to protect the transportation unit (e.g., fillers, caps, corner posts, stretch wrap). Such efforts minimize materials that are put in landfills and minimize overall energy use. Further, co-packing the products employs people and avoids designing costly equipment that may have a limited lifespan due to the specialized nature of the packaging.

Transportation and warehousing are the most frequent places where damage may occur. Compression can occur with static and dynamic loads. Static loads, such as those induced by stacking in a warehouse, are affected by load, time, humidity (static and cyclic), stack pattern, and pallet type and condition. Dynamic loads, such as those induced in transit, are affected by momentary loading, clamp handling, railcar humping/coupling, and trailer sway. In addition to compression, shock (drop, impact, falls), vibration, puncture, temperature, humidity, pressure, corrosion, energy, contamination, shrink, and time are contributors to damage.

Transport packaging has been historically viewed as a value retainer as opposed to a value adder. This approach is being challenged by increased contributions from transport packaging such as shelf presence, marketing, advertising, and ongoing consumer uses, including product dispensing and disposal.

For the packaging of the present disclosure, numerous precautions have been taken to ensure that the storage bin 110, which is marketed as product, arrives at the retailer in pristine condition and maintains its integrity with consumer use in the home environment. Storage bins 110 are not shipped inside of other shipping or corrugated boxes. To ensure the integrity of the storage bins 110, several variables are controlled and include double wall corrugate, shrink wrap to prevent rubbing and scuffing and to provide friction between packages while stacked, corner posts to prevent vertical crushing, top and bottom caps and slip sheets to distribute load and protect exposed edges, load securement tools in trucks, proper truck handling such as the use of forklifts only without clamping, pallet utilization above 90 percent, pallet quality such as no broken deck boards, stretch wrapping extended to the bottom of the pallet to prevent unit load shifting, limiting stacks to two-high instead of four-high, and direct shipping from co-packer to retailer distribution centers and stores to prevent additional handling.

In another aspect of the present invention, interior packaging such as another box, forms, fillers, dividers, bags, and/or cartons can be used to strengthen the storage bin 110 and further minimize susceptibility to damage in transit or use. In still another aspect of the present invention, personal care products can be shipped to a co-packer in plain packaging, which the co-packer simply slips into a storage bin 110 to minimize product handling.

A potentially key factor in making components of the packaging system 100 conducive to reuse is the design of those components. For example, a consumer is more likely to reuse the storage bin 110 if the storage bin 110 is attractive enough to complement the consumer's home décor and sturdy enough to last. Features of the packaging system 100 that enhance its appeal include premium branding graphics that are of high quality and provide an abundance of “pop.” In addition, graphics can cover the front, top, and back panels of the storage bin 110, as multi-sided branding is important for bill boarding. Also, a cover 120 that stays in place atop the storage bin bottom 115 helps to keep everything in the storage bin 110 and helps to keep people out. In addition, both the cover 120 and any bin enclosures 165 should be easy for a consumer to remove and should be removable without destroying the storage bin 110 or its usefulness.

To add interest and thus increase the chance of reusing components of the packaging system 100, the storage bin 110 can be provided with a bin design. The bin design refers primarily to the design of the storage bin 110, including its shape, its coloring, and the graphics associated with it. The bin design can be associated with a color, a pattern, a holiday, a season, a character, a story, an action, an event, or any other suitable subject matter or combination of these. If a character, bin designs include a fictional character, a non-fictional character, and an animated character such as a cartoon character. If an event, bin designs include events such as toilet training or a specific holiday. Graphics described throughout this disclosure can include baby-related, kitchen-related, and age-related graphics, and other graphics that are attractive to a caregiver or a baby. In an example, a bin design can include a cartoon character, where the graphics placed on the personal care product relate to that cartoon character. Graphics can be depicted on the personal care product by printing, embossing, engraving, or by any other suitable means. The design can include a label area 180 to allow the consumer to label the storage bin 110.

The features selected for the storage bin 110 are selected to be representative of the reuse of the storage bin 110. Such features include the label area 180 and the cover 120, along with other features described herein. Such features ensure that the storage bin 110 functions properly during reuse and complements the user's home décor. Appropriate marriage of graphics and structure will guide the user selecting a use for the container. For example, cartoon graphics are usually more appropriate for a toddler's bedroom or playroom, while home décor graphics such as basket weave, florals, etc. are more appropriate for an adult's bedroom or the family living area, and baby-related graphics are more appropriate for a nursery.

The retail environment requires packaging to do much more than just store its contents; the package must sell the product. The graphics of the bin design, which include colors, images, and text, must be chosen with the potential end consumer constantly in mind. The selection of storage bin graphics is based on many factors including baby décor trends, home décor trends, color trends, and storage container trends.

Depending on the sophistication of printing desired, how many storage bins are needed, and the magnitude of the investment, a variety of printing processes ranging in low to high quality are available and include printing directly onto combined board, preprinting the linerboard, single-face laminating, and labeling. Specific processes include flexo postprint, CHROMAPAK color printing available from Weyerhaeuser Company, flexo advanced postprint, flexo premium postprint, flexo preprint, litho label, singleface lamination, and digital ink jet. These differ primarily in the number of colors and in line colors versus process colors versus spot colors. Corrugated graphic substrates can range from low to high end paper, including kraft, mottle white, bleached white, and clay-coated white.

In one aspect of the present disclosure, the storage bins 110 are printed using flexographic preprint. Other aspects of the present disclosure use offset lithography, digital printing, and direct printing. Printing methods can be leveraged to differentiate graphics by retailer, brand, product line, promotion, etc. In still other aspects of the present disclosure to maintain differentiation and bring innovation to the storage bin 110, 6-color offset, 150-175 line screen or higher can be used.

In other aspects of the present disclosure, further customization can be achieved with specialty decorative finishes and effects such as pearlescence, metallic inks, hot foil stamping, fluorescent inks, fragrance inks and adhesives, embossing, varnish, glitter, crackle, mirror, color change, matte, mesh, sparkling, soft-touch, flocking, lenticular, and holographics. The generous use of these special finishes or special effects can provide maximum visual “pop” and sensory appeal.

To add further interest and thus increase the chance of reusing components of the packaging system 100, the bin enclosure 165 can be provided with an enclosure design. The enclosure design refers primarily to the design of the bin enclosure 165, including its shape, its coloring, and the graphics associated with it. The enclosure design can be associated with a color, a pattern, a holiday, a season, a character, a story, an action, an event, or any other suitable subject matter or combination of these, such as those described above with respect to the bin design. For example, an enclosure design can be a cartoon character, where one or more of the graphics placed on the bin enclosure 165 and/or the shape of the bin enclosure 165 relate to that cartoon character. Graphics can be depicted on the bin enclosure 165 by printing, embossing, engraving, or by any other suitable means or combination of these.

To further optimize interest and thus increase the chance of reusing the components of the packaging system 100, the inner package 160 can be provided with a package design. The package design refers primarily to the design of the inner package 160, including its shape, its coloring, and the graphics associated with it. The package design can be associated with a color, a pattern, a holiday, a season, a character, a story, an action, an event, or any other suitable subject matter. For example, the package design can include a cartoon character, where the graphics placed on the inner package relate to that cartoon character. Graphics can be depicted on the inner package by printing, embossing, engraving, or by any other suitable means or combination of these.

As used herein, the term “same as” refers to graphics or other thematic elements that depict a common character, shape, and the like. One graphic can be a different size and can have subtle differences and still be considered the same as a second graphic. For example, a large star graphic would be the same as a small star graphic because both depict a star. In another example, the graphic of a cartoon character clapping would be the same as a graphic of the cartoon character running because both depict the same cartoon character.

In another aspect of promoting reuse of the packaging system 100, the removable bin enclosure 165 includes product-identifying graphics 195. Product-identifying graphics 195 can include the type of personal care product disposed in the interior space 125, the size or sizes of personal care products disposed in the interior space 125, and the count, amount, or volume of personal care products disposed in the interior space 125, all as applicable to the type of personal care products. Product-identifying graphics 195 can also include the manufacturer, customer service information, use instructions, patent markings, and any other information typically disposed on a package of personal care products.

To further enhance a consumer's interest in reusing the storage bin 110, the storage bin 110 includes non-product-identifying graphics 190 such as those described above. The non-product-identifying graphics 190 on the storage bin 110 can be registered in relation to the storage bin bottom 115. The non-product-identifying graphics 190 on the storage bin 110 can include graphical elements such as labels, hinges, hasps, locks, and other elements to enhance the look of the storage bin 110. The non-product-identifying graphics 190 can further include textured elements such as those that can be supplied by embossing.

In another aspect of promoting reuse of the packaging system 100, the storage bin 110 defines an interior surface 185, and the interior surface 185 includes non-product-identifying graphics 190 of the types described above.

The attractiveness of reusing the packaging system 100 can be further enhanced by providing an array of different but coordinated or complementary storage bins 110 and packaging systems 100. In general, the personal care products disposed in a first storage bin 110 differ in count, size, or type, for example, from the personal care products disposed in a second storage bin 110. Consequently, the first storage bin 110 differs from the second storage bin 110 in their non-product-identifying graphics, in their shapes, in their sizes, in any other suitable attribute, or in some combination of these. The first storage bin 110, however, does not differ from the second storage bin 110 enough that they are no longer coordinated or complementary.

For example, a first storage bin 110 including personal care products of a first size can be sold in conjunction with a second storage bin 110 including personal care products of a second size. The first storage bin 110 will exhibit different non-product-identifying graphics 190 from the second storage bin 110, but the non-product-identifying graphics 190 of one will complement the non-product-identifying graphics 190 of the other in that their color, pattern or other schemes will be complementary. Alternatively, the first storage bin 110 will be of a different size from the second storage bin 110, but the second storage bin 110 will be able to nest in the first storage bin 110. Alternatively, the first and second storage bins 110 will have different volumes, but will have the same size covers 120 and/or will be able to stack neatly. Again, these are merely examples of the potential spectrum of product differences and storage bin differences available to a manufacturer or retailer and therefore to a consumer.

In another example, the personal care products in a first storage bin 110 are of a different type from the personal care articles of a second storage bin 110. The first and second storage bins 110 can again differ by non-product-identifying graphics 190, size, shape, or other attribute as long as the first and second storage bins 110 are still complementary. It should be noted that these examples do not exclude third, fourth, and additional personal care product types, sizes, counts, etc. in third, fourth, and additional storage bins 110 that are each different but complementary.

In still another example, a manufacturer can supply trademarked personal care products of a first type, size, count, etc. to a first retailing company (i.e., one using a first brand name) in a first storage bin 110, and to a second retailing company (i.e., one using a second brand name) in a second storage bin 110. In addition, the first and second storage bins 110, although different in the ways described herein, can still be complementary in the ways described herein. Further, one or both of the first and second storage bins 110 can also carry the name of the retailing company selling it.

Retailer support can be an important factor in attracting sales as well. Retailer differentiation is one way to enhance retailer support, and can be achieved in many ways. For example, retailer differentiation can be achieved via graphics such as colors, patterns, etc.; structure such as substrates, dimensions, shape, storage bin type, etc.; special effects such as holographics, flocking, fragrance, etc.; in-store displays such as storage bin graphics creating a billboard with a single- or mixed-pallet design. Transportation units can be customized for a retailer or within a retailer to accommodate the range of very large to small stores.

In addition, graphics can be different on a number of packaging systems 100 to make up a set to encourage multiple purchases. Graphics can be coordinating in a general complementary sense such as similar colors and/or patterns, or can be highly-coordinating whereby stacking the packaging systems 100 or storage bins 110 in a vertical or horizontal orientation in a designated order makes a picture or scene appear across the exposed sides 135. Further, graphics and storage bin design can be selected to cover promotions across all or some of the personal care product categories described herein including facial tissue, bathroom tissue, absorbent articles, etc.

Retailer differentiation can be achieved through unique package size, product count, and price point. Retailers can get special promotions and displays, which also provide differentiation. Promotions are often at a lower price versus everyday pricing and/or include a giveaway such as a CD or a trial pack of wet wipes. The present disclosure allows differentiation within a brand, particularly a manufacturer's brand, whereas differentiation was previously only between a manufacturer's brand and a retailer brand. As a result, packaging, merchandising, and distribution design and execution can be customized to accommodate each retailer.

These and other modifications and variations to the present disclosure can be practiced by those of ordinary skill in the art, without departing from the spirit and scope of the present disclosure, which is more particularly set forth in the appended claims. In addition, it should be understood that aspects of the various aspects can be interchanged both in whole or in part. Furthermore, those of ordinary skill in the art will appreciate that the foregoing description is by way of example only, and is not intended to limit the disclosure so further described in such appended claims.

What is claimed is:

1. An array of packages adapted for transporting and selling personal care products, the array comprising:
a first package including first removable product-identifying graphics and a plurality of first personal care products disposed within a first storage bin, the first storage bin including a first design and being substantially free of product-identifying graphics; and
a second package including second removable product-identifying graphics and a plurality of second personal care products disposed within a second storage bin, the second storage bin including a second design and being substantially free of product-identifying graphics, wherein the second design is different from and complementary to the first design.

2. The array of claim 1, wherein the first and second personal care products are different sizes of the same product.

3. The array of claim 1, wherein the first and second personal care products are different products.

4. The array of claim 1, wherein the first and second personal care products are the same product, and wherein the first and second packages are adapted to be sold at differently-branded retailers.

5. The array of claim 1, wherein the first and second personal care products are different combinations of personal care products.

6. The array of claim 1, wherein the first and second product-identifying graphics are identical.

7. The array of claim 1, wherein the first personal care products are disposable absorbent articles.

8. The array of claim 1, wherein the first personal care products are diapers.

9. The array of claim 1, wherein the first and second personal care products are the same product.

10. The array of claim 1, wherein the first design is a seasonal design, and wherein the second design is a non-seasonal design.

11. The array of claim 1, wherein the first and second storage bins are free of product-identifying graphics.

12. The array of claim 1, further comprising a third package including third removable product-identifying graphics and a plurality of third personal care products disposed within a third storage bin, the third storage bin including a third design and being substantially free of product-identifying graphics, wherein the third design is different from and complementary to each of the first and second designs.

13. An array of personal care product packages, the array comprising:
a first package including a plurality of personal care products of a first size, wherein the first package includes a first storage bin having first non-product-identifying graphics and being substantially free of product-identifying graphics; and
a second package including a plurality of personal care products of a second size, wherein the second size is larger than the first size, wherein the second package includes a second storage bin having second non-product-identifying graphics and being substantially free of product-identifying graphics, and wherein the second non-product-identifying graphics are different from and complementary to the first non-product-identifying graphics.

14. The array of claim 13, wherein the non-product-identifying graphics are complementary by color.

15. The array of claim 13, wherein the non-product-identifying graphics are complementary by pattern.

16. The array of claim 13, wherein the first storage bin includes a first cover removably attached to the first storage bin, wherein the second storage bin includes a second cover removable attached to the second storage bin, and wherein the first cover is sized to fit on the second storage bin and the second cover is sized to fit on the first storage bin.

17. An array of personal care product packages, the array comprising:
a first package including a plurality of personal care products of a first type, wherein the first package includes a first storage bin having first non-product-identifying graphics and being substantially free of product-identifying graphics; and
a second package including a plurality of personal care products of a second type, wherein the second type is different from the first type, wherein the second package includes a second storage bin having second non-product-identifying graphics and being substantially free of product-identifying graphics, and wherein the second non-product-identifying graphics are different from and complementary to the first non-product-identifying graphics.

18. The array of claim 17, further comprising a third package including a plurality of personal care products of a third type, wherein the third type is different from the first and second types, wherein the third package includes third non-product-identifying graphics, and wherein the third non-product-identifying graphics are different from and complementary to the first and second non-product-identifying graphics.

19. An array of personal care product packages, the array comprising:
a first package including a first plurality of personal care products carrying a trademark, wherein the first package includes a first storage bin having first non-product-identifying graphics and being substantially free of product-identifying graphics, and wherein the first package is provided to a first retailer; and
a second package including a second plurality of the personal care products carrying the trademark, wherein the second package includes a second storage bin having second non-product-identifying graphics and being substantially free of product-identifying graphics, wherein the second package is provided to a second retailer, wherein the second non-product-identifying graphics are different from the first non-product-identifying graphics, and wherein the first retailer has a brand name different from a brand name of the second retailer.

20. The array of claim 19, wherein the first and second storage bins each have a shape, and wherein the first storage bin shape is different from the second storage bin shape.

21. The array of claim 19, wherein the first and second pluralities of personal care products each have a count, and wherein the first plurality count is different from the second plurality count.

22. The array of claim 19, wherein the brand name of the first retailer is displayed on the first package.
